(19) United States
(12) Patent Application Publication
(10) Pub. No.: US 2010/0209918 A1
(43) Pub. Date: Aug. 19, 2010

(54) SYSTEM SUBSTITUTE PCR

(75) Inventors: Hong Jiang, Beijing (CN); Tongbing Liao, Beijing (CN); Bisheng Jiang, Beijing (CN)

Correspondence Address: BUCHANAN, INGERSOLL & ROONEY PC, POST OFFICE BOX 1404, ALEXANDRIA, VA, 22313-1404, US

(73) Assignee: SYSTEM SUBSTITUTE PCR

(21) Appl. No.: 12/600,063

(22) PCT Filed: May 9, 2008
(86) PCT No.: PCT/CN08/70928

§ 371 Date: Nov. 13, 2009

(30) Foreign Application Priority Data
May 17, 2007 (CN) 200710107828.7
Nov. 2, 2007 (CN) 200710176760.8

(51) Int. Cl. C12Q 1/68

(52) U.S. Cl. 435/6

(57) ABSTRACT

The present invention relates to the substitute PCR of molecular diagnosis through the direct test-gene PCR substituted with indicator-DNA amplification. The present art has developed a new concept that the test-DNA is translated into the indicator-DNA following the indicator-DNA indirect PCR. In one aspect of this substitute PCR involves the test DNA-indicator translation or substitution by hybridization-ligation strategy, in which the two adjacent test sequences as double-probes are added to two ends of the indicator following indicator ends associated with test DNA through added the ends sequences. By the test DNA association help, two ends of the same indicator that added ends sequences will be closer and come together, and the nick between ends will be jointed by Taq-ligase, and the indicator will become circular DNA. Another aspect of the invention includes the reverse amplification of circular indicator by using center sequence of indicator as reverse PCR primers. Such substitute PCR efficiency is usually lower than the direct test PCR. Therefore it limits the system sensitivity and reduces the cross-contamination amplification.

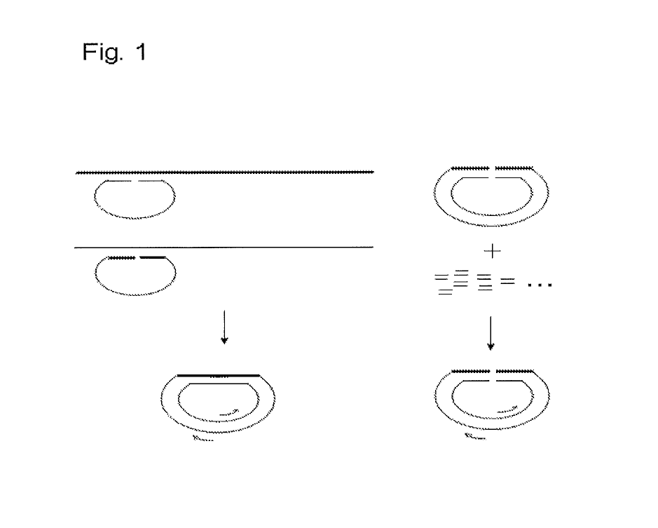

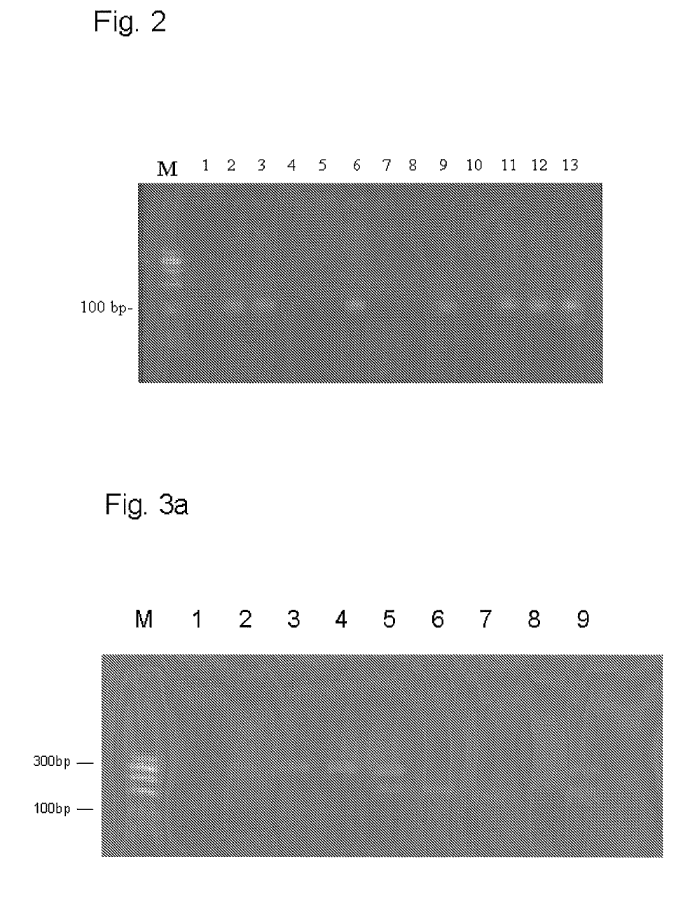

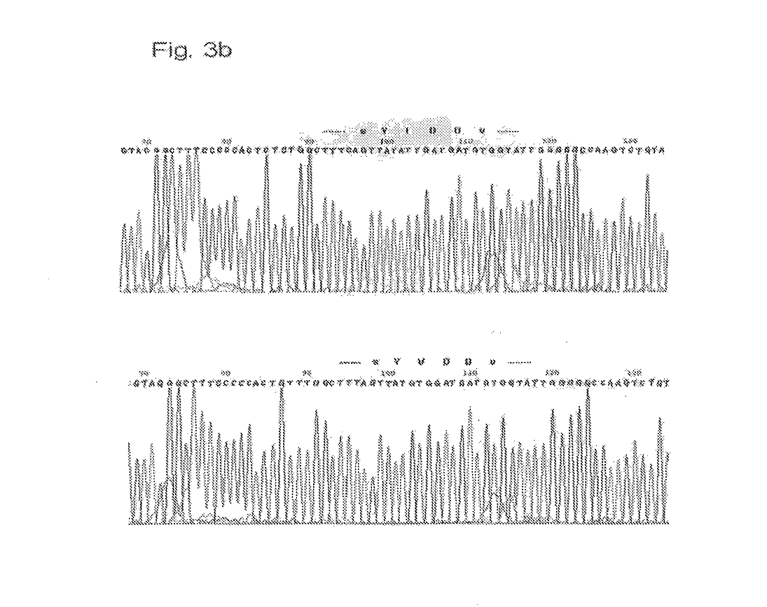

### FIELD OF THE INVENTION

The present invention relates to the field of molecular diagnosis by using Polymerase Chain Reaction (PCR). And more especially, it relates to a method of substituting the test PCR with indicator DNA amplification.

### BACKGROUND OF THE ART

One Friday night of 1983, Kary B Mullis unusually created a very brilliant idea of DNA duplication or amplification in the test tube (The unusual origin of the polymerase chain reaction, Sci. Am. 262: 56, 1990; and U.S. Pat. No. 4,683,202). From that, the PCR method is continuously improved by Cetus, PE, Roche company and so on. Since Taq polymerase that used in the PCR was isolated from a colony of Thermus Aquaticus collected in a hot spring at Yellowstone National Park (Saiki. R. K., et al, Science 239:p 487-491, 1988), the PCR technique became a possible, powerful and automatic method. Now the PCR techniques are widely used in gene cloning, DNA Sequencing, molecular diagnosis and so many fields. It is generally considered as the most important techniques of modern molecular biology. The scientists also have developed many PCR based application methods and hundreds of PCR improved protocols (Molecular Cloning, 3^{rd} Edition).

The current biology has entered the time of functional-molecules research post the genome era after the human genome project. The molecular diagnosis or test by PCR has all the more broader applications. It has penetrated into every field of biology. Since the PCR was first used in the clinical test in 1989, the PCR, which has extreme sensitivity advantage and simplest operation, became the basic method for many clinical tests that include two areas: the detection of infectious disease organisms, especially the ones that are difficult or impossible to culture; and the detection of variations and mutations in genes, especially the genes for genetic diseases, cancer and heart disease. However, the PCR exponentially amplification method is too sensitive, and the twentieth power of two templates is about one million amplification (2²⁰=1 million). Due to the non-specific amplification of the contamination of amplicon aerosol and cross contamination of sample, the major problem of PCR clinical application is false positive reaction test. The current approaches to control the contamination include: using Lamina flow cabinet, barrier pipet tips, fresh gloves, aliquot PCR reagent, ultraviolet (UV) irradiation and separated laboratory. The further advanced PCR method was designed by destroying the PCR products (amplicons) via PCR substrate dTTP replaced with dUTP and the Uracil-DNA-glycosylase treatment (Hartley J. L. et. al, 1993 PCR Methods App 1.3: s10-s14). These methods are truly helpful, but they are cumbersome operations that cannot completely prevent contamination for the extremely sensitive PCR methods

### DESCRIPTION OF THE EMBODIMENTS

The purpose of preventing PCR system contamination, the present invention System Substitute PCR (ssPCR) replaced the direct test PCR with the indicator PCR system. The critical feature is that the regular PCR is just a simple step for amplifying objective DNA as a working PCR. For example, if people want to test “A” gene, just amplification “A” DNA is enough. The present art ssPCR has developed this traditional concept into that the test gene template is substituted for the indicator DNA followed by the indicator DNA amplification. The test PCR substituted with the indicator PCR has two major privileges: First, the ssPCR efficiency of only once substitution is much lower than that of the test DNA direct amplification. And the efficiency of substitution of ssPCR is easily adjusted by setting different conditions (thermal-ligase cycling). Therefore, the sample cross contamination or/and the amplicon aerosol contamination is impossible to be translated into the indicator, so that the substitution prevents background amplification of the cross-contamination. Second, the one test DNA can substitute to a series of different (irrelevant), independent indicator DNA system1, 2, 3 - - - . When indicator DNA system1 PCR is contaminated, we can immediately change it to a different indicator DNA, such as indicator DNA 2 system, or indicator DNA3, 4 - - - system and so on.

In one aspect of the present invention, the test DNA is substituted into indicator DNA. The indicator DNA is a 80-150 bp DNA fragment which sequence is irrelevant with the test sequence (definition: the continuous 6-8 bases or more nucleotide with the same sequences between two DNAs are relationship, otherwise are irrelevant). In another aspect of the present invention, we choose a 30-50 bp sequence of test DNA as Preserved Hybridization Region (PHR), in which the right half of PHR is linked with upstream (5′) end of indicator DNA as a cap, the left half of PHR is attached to downstream (3′) end of indicator DNA as a tail. These indicators DNA with cap and tail that have test Preserved Hybridization Region can be associated with test template through their complementary cap and tail sequences. With the test template association help, the indicator DNA cap end will be near or closer to tail termini and form the hybridization of the cap and tail sequence of indicator with test strands. The nick between cap and tail ends of hybrids is joined by Taq ligase. Therefore, the indicator that is associated with the test becomes the circular DNA and then the circular indicator DNA can be reversely amplified (reverse PCR) by using the center sequence of indicator as reverse primers. The indicator without test hybridization help is still linear DNA and cannot be reversely amplified. As a result, only the positive test can be substituted into the circular indicator DNA following reverse indicator PCR. Therefore the background contamination cannot be translated into indicator and non-specific amplification is effectively avoided.

### 1. Preparation of Indicator DNA:

The polynucleotide that is irrelevant with test genes is chosen as indicator DNA through the gene data base or Blast search. The definition of irrelevance is that two genes contain less than 6-8 base continuous same nucleotide sequences between test and indicator. The length of indicator DNA is from 80 base-pair to 1000 bp, it is better and more convenient to use 80 to 500 bp for the agarose-gel check and optimum PCR efficiency, and it is better and more convenient to use 80 to 150 bp for the agarose-gel check and optimum PCR efficiency. For the test template specifically replaced by the indicator DNA, the part of test sequence is selected as Preserved Hybridization Region (PHR), which is 20-200 bp length of specific hybridization sequence, it is better and more convenience from 30 to 50 bp length. The PHR is divided into two parts. The right part is added to the upstream (5′) end of indicator as the probe cap. The left part is attached to the downstream (3′) end of indicator as the probe tail. The cap fragment plus the first about 20 bp sequences of 5′ end of indicator are selected as Forward primer of indicator DNA preparation by using the sense chain sequence. The tail fragment with last about 20 bp sequence of downstream (3′) end of indicator to use their antisense chain sequence as reverse primer of indicator DNA preparation. By using this pair of phosphated primer and pfu enzyme with 3′-5′exonuclease activity, the blunt end indicator DNA with cap and tail is amplified and prepared, and further purified by using PCR agarose gel purification Kit of Qiagen Inc. (Cat No. 28604).

### 2. Hybridization-Ligation Reaction of the Test-Indicator.

The hybridization of indicator DNA (which has the probe cap and tail) with the test gene template is performed through base paring between the complementary sequence of the indicator probing end and Preserved Hybridization Region (PHR) of the test. With help of positive test DNA, the indicator cap end will be near or adjacent to same indicator tail end through association of test PHR with indicator ends. The 5′ end phosphate of indicator cap and 3′ end hydroxyl group of same indicator tail come together and be juxtaposed each other. The nick of cap phosphate group end and hydroxyl group termini are ligated to form a phosphodiester bond by Thermus ligase. Therefore, the indicator DNA that is hybridized with the positive test strand will become the circular indicator DNA molecules. However, without positive test DNA hybridization help, the blunt end of the same indicator double strands in the negative test samples has no chance for the ligation of same indicator cap with tail. The ligation rate between the different indicator molecule ends is much lower than that of one nick of double-strands of test-indicator hybridization. These blunt end ligations of different indicator blunt are further completely inhibited by adding excess irrelevant double strands of the oligo inhibitor that is 8-20 bp blunt end double strands of DNA. The specific nick ligation will occur only if the sequences are perfectly paired to the complementary test-indicator hybrids and have no gaps between them. Therefore, a single-base nucleotide mutation can be detected.

Through decreasing the ratio of indicator DNA with test template and increasing the temperature of hybridization-ligation reaction, ssPCR can efficiently adjust the sensitivity of test-indicator substitution and therefore further control the nonspecific background amplification.

By using Ligase Chain Reaction (Barany F., PNAS 1991, 88:p 189-193), The 2-30 cycles of thermal ligation reactions assist the following cycles of the nick ligation through the circular DNA that is made in the first cycle as template of the following cycles. The present art can further to increase the sensitivity of tiny amounts of test DNA.

### 3. Reverse PCR of Circular Indicator DNA

The indicator DNA PCR amplification is designed to reverse amplification by using the center sequences of indicator DNA as reverse PCR primers. In the middle of indicator, the reverse primers bind to the indicator center and elongate toward the cap and tail direction of indicator. Therefore, the circular indicator DNA can be exponentially amplified by reverse PCR. The indicator DNA reverse PCR just indirectly reflects the test DNA measurement. The linear indicator DNA cannot be reverse amplified by using center sequence primer. There is only linear increasing of half-length of indicator in the negative test. As indicator varies, the indicator DNA can be designed to break or separate in the center region. In another word, it can make two separated Right indicator and Left indicator, the right PHR and left PHR are still made for the cap and tail end, the other separated ends sequences of indicator are used as reverse primers. Unfortunately, this case is harder for the indicator DNA preparation and purification.

The examination of reverse PCR is accomplished by loading to agarose gel check or by adding fluorescent dye SYBR Green/Molecular Beacon/Taqmen/Light Cycler for Real-Time PCR.

### THE ADVANTAGES OF PRESENT INVENTION

(1) The test PCR is substituted with the indicator PCR, which avoids the cross contamination of only one PCR system. If one system failed, another PCR system could be used.
(2) The Present Invention expanded the sensitivity range of traditional PCR through decreasing or increasing the efficiency of ssPCR.
(3) Both DNA or RNA of test can be substituted into indicator DNA. So, RNA test needn't Reverse-Translated to cDNA and can be directly tested.
(4) The single base substitution or genetic mutation of genes can be efficiently analyzed by the ssPCR method.
(5) The Real-Time PCR of ssPCR coupling with fluorescent probe is more efficient, more sensitive quantity analysis and high-through put analysis of genes than that of Southern-Blot and Northern-Blot protocols.

### Operation Protocol

1. Preparation of Indicator DNA with Cap & Tail:

### (1) Indicator Primer Synthesis

Forward primer: The sense chain sequence of the right part PHR of test gene plus the upstream end about 20 bp of indicator DNA as 5′end preparation primer.

Reverse primer: The antisense chain sequence of the left part PHR of test gene plus the downstream end about 20 bp of indicator DNA as 3′end preparation primer.

The primer oligos are synthesized by using the solid phase phosphate-triester method followed by the phosphoration of oligo 5′ end. The phosphated oligo also can purchased from the commercial oligo synthesis company.

(2) Amplification of Indicator DNA with Cap & Tail

| | | |
|---|---|---|
| The irrelevant DNA | 1 | μl |
| (which part sequence as indicator) | | |
| Forward primer(5 μM) | 1 | μl |
| Reverse primer(5 μM) | 1 | μl |
| 10 mM dNTP | 1 | μl |
| 10X pfu buffer | 5 | μl |
| pfu | 1 | μl |
| dH₂O | 40 | μl |
| Total | 50 | μl |

First set in 95° C. to denature for 5 minutes, and then 25 cycles of 94° C. 30 seconds, 54° C. annealing 30 seconds, 72° C. elongation 35 seconds, and after 25 cycles, the final 72° C. for 10 minutes.

The PCR products are loaded to the 1.5%-2.0% agarose gel for gel purification by using the Qiagen Inc kit.

### 2. Synthesis of Oligo Inhibitor:

The extremely rare sequences, for example the recognition sequence of Homing Endonuclease, are selected as the Oligo Inhibitor which is irrelevance with test and indicator (Definition: the continuous 4-6 base and more is the same sequence as relationship, otherwise as irrelevance). The Oligo Inhibitors are used for the inhibition of non-specific ligation between different indicator ends. The 8-20 base of rare sense chain oligo and antisense chain oligo are separately synthesized by the commercial supplies. Following both oligos are combined by 95° C. denature and qick in ice.

### 3. Synthesis of Reverse PCR Primers:

The present art ssPCR takes the about 40 bp sequence of the indicator center region into two parts, in which the right half 20 bp of sense chain sequence is as 5′end Reverse PCR Primer, and the left half 20 bp of antisense chain sequence as 3′end Reverse PCR Primer.

4a. Purification of the Test DNA:

The test sample is first extracted once with equal of phenol/chloroform/isopentyl alcohol (25:24:1) reagent and once with chloroform alone to remove the proteins.
(1) Take test sample in the 100 μl volume in EP tube.
(2) Add 100 μl of phenol/chloroform/isopentyl alcohol mix, vortex.
(3) Spin 1500 rpm×5 minutes in micro-centrifuge.
(4) Transfer supernatant to a fresh tube, add 100 μl of chloroform and vortex.
(5) Spin 5 minutes at high speed in microcentrifuge.

The supernatant DNA further purification is accomplished by using Qiagen Inc DNA purification column kit according the operation menu,

4b. RNA Isolation

Single-step of Guanidinium Method for RNA isolation (Chomczynski, P. et, al. 1987 Anal. Biochem. Vol 162, 156-159) is used for RNA substituted to indicator followed by indicator amplification.

The test sample is lysed in 1 ml of Trizole (0.5 ml of 4 M guanidinium, 0.5 ml of phenol and 0.05 ml of sodium acete pH 4.0). The viscosity of the solution is reduced by drawing the lysate through a 20-G needle. After adding 0.2 ml of chloroform and spinning 5 minutes in microcentrifuge, the solution will be separated to two layers. The supernatant is added 0.5 ml (equal volume) of isopropanol set in −20° C. for more than 2 hours followed by spinning and washing once with 70% ethanol. The final precipitated RNA pellet is dissolved with 50 μl of DEPC treated water (dH₂O).

### 5. Hybridization-Ligation Reaction of the Test-Indicator:

| | | |
|---|---|---|
| Purified test sample | 1 | μl |
| Purified indicator with cap & tail | 1 | μl |
| 10X Ligase buffer | 2 | μl |
| Oligo inhibitor (0.1 mM) | 1 | μl |
| Thermus Ligase(such as Taq ligase) | 0.5-1 | μl |
| dH₂O | 14 | μl |
| Total | 20 | μl |

Set in 95° C. to denature for 5 minutes, and then in 50° C. (40-70° C.) hybridization-ligation for 10 minutes.

For decreasing the sensitivity of substitution of test to indicator, increase the ratio of test with indicator and temperature of ligation. For increasing the sensitivity of substitution, take 2-30 cycles of 95° C. to 50° C. thermal-cycling of denature to hybridization-ligation.

### 6. Reverse PCR of the Circular Indicator:

Use the solution of hybridization-ligation reaction of the test-indicator as the reverse PCR template and the center sequences of indicator (Reverse primer F, Reverse primer R) as the reverse PCR primers.

| | | |
|---|---|---|
| Solution of hybridization-ligation | 1-2 | μl |
| of the test-indicator | | |
| 5′end Reverse pimer F(5 μM) | 1 | μl |
| 3′end Reverse pimer R(5 μM) | 1 | μl |
| 10 mM dNTP | 1 | μl |
| 10X Taq PCR buffer | 5 | μl |
| Taq polymerase | 1 | μl |
| dH₂O | 40 | μl |
| Total | 50 | μl |

First set in 95° C. to denature for 5 minutes, and then 25-30 cycles of 94° C. denature 30 seconds, 54° C. annealing 30 seconds, 72° C. elongation 35 seconds, and after 25-30 cycles, the final 72° C. for 10 minutes.

### 7. Analysis of PCR Products:

Load about 20 μl of PCR products to 1.5-2.0% agarose gel for electrophoresis check. Or add fluorescent dye SYBR Green/Molecular Beacon/Taqmen/Light Cycler for Real-Time PCR.

### Example 1

### Test of Hepatitis B Virus HBsAg

The HBV detection by using S gene PCR approaches is widely applied in the clinical test and confirmed diagnosis. However the major limitation of contamination hampered their further application as a routine way. The present invention ssPCR avoids the cross contamination between samples and re-contamination of amplicons by system substitution.

1. Take 13 clinical blood sample, which 7 positive hepatitis and 6 negative tested by the KEHUA company anti-HBsAg antibody ELISA Kit.

The 13 DNA samples are first extracted once with equal of phenol/chloroform/isopentyl alcohol (25:24:1) reagent and once with chloroform alone to remove the proteins.

Spin 5 minutes at high speed in micro-centrifuge.

The supernatant DNA further purification is accomplished by using Qiagen Inc DNA purification column kit according the operation menu.

2. Preparation of Indicator DNA:

(1) Select parasite Gluthione S-Transferase (GST) genes initial 100 bp sequence

5′-cctatac taggttattg gaaaattaag ggccttgtgc
aacccactcg acttcttttg gaatatcttg aagaaaaata
tgaagagcat ttgtatgagc gcg-3′

as indicator DNA template.

(2) Select the HBV surface antigen amino acid 124-138 gene sequence

5′-gc acg att cct gct caa gga acc tct atg ttt ccc
tct tg-3′
3′-cg tgc taa ggt cga gtt cct tg-5′

as Preserved Hybridization Region.

(3) Synthesis of Indicator 5′end primer: The 18 base sense chain sequence of right half PHR of test HBsAg gene plus the upstream end 18 base sense chain sequence of indicator GST DNA.

5′-c tct atg ttt ccc tct tg cctatac taggttattg
g-3′

(4) Synthesis of Indicator 3′end primer: The 22 base anti-sense chain sequence of left half PHR of test HBsAg gene plus the downstream end 19 base anti-sense chain sequence of indicator GST DNA.

5′-gt acc ttg agc tgg aat cgt gc cgc gctcatacaa
atgctc-3′

(5) Amplification and Purification of indicator DNA with cap and tail

| | | |
|---|---|---|
| pGEX-2T plasmid DNA | 1 | μl |
| (which part GST sequence as indicator) | | |
| Forward primer(5 μM) | 1 | μl |
| Reverse primer(5 μM) | 1 | μl |
| 10 mM dNTP | 1 | μl |
| 10X pfu buffer | 5 | μl |
| pfu | 1 | μl |
| dH₂O | 40 | μl |
| Total | 50 | μl |

First set in 95° C. to denature for 5 minutes, and then 25 cycles of 94° C. 30 seconds, 54° C. annealing 30 seconds, 72° C. elongation 35 seconds, and after 25 cycles, the final 72° C. for 10 minutes.

The PCR products are loaded to the 1.5%-2.0% agarose gel for gel purification by using Qiagen Inc kit. Final indicator DNA elute in 50 μl dH₂O.

3. Synthesis of Blunt End Oligo DNA Inhibitor

Take rare and irrelevant sequence 5′-GCGGTACCGG-3′ and which complement anti-sense sequence 5′-CCGGTACCGC-3′ for commercial synthesis.

combine them, 90° C. heat 5 minutes and cool at Room-Temperature, store at −20° C.

4. Synthesis of Reverse Primers

Take the center region of indicator GST DNA

5′-gtgc aacccactcg acttcttttg gaatatcttg-3′
3′-cacg ttgggtgagc tgaag-5′

the right part 19 base sense-chain sequence 5′-cttcttttg gaatatcttg-3′ as 5′end Reverse primer; the left part 19 base anti-sense chain sequence 5′-gaagt cgagtgggtt gcac-3′ as 3′ end Reverse primer.

5. Hybridization-Ligation Reaction of the Test-Indicator:

| | | |
|---|---|---|
| Purified test sample | 1 | μl |
| Purified indicator with cap & tail | 1 | μl |
| 10X Ligase buffer | 2 | μl |
| Oligo inhibitor (0.1 mM) | 1 | μl |
| Thermus Ligase(such as Taq ligase) | 0.5-1 | μl |
| dH₂O | 14 | μl |
| Total | 20 | μl |

Set in 95° C. to denature for 5 minutes, and then in 50° C. (40-70° C.) hybridization-ligation for 10 minutes.

6. Reverse PCR of the Circular Indicator:

Use the solution of hybridization-ligation reaction of the test-indicator as the reverse PCR template and the center sequences of indicator (Reverse primer F, Reverse primer R) as the reverse PCR primers.

| | | |
|---|---|---|
| Solution of hybridization-ligation | 2 | μl |
| of the test-indicator | | |
| 5′end Reverse pimer F(5 μM) | 1 | μl |
| 3′end Reverse pimer R(5 μM) | 1 | μl |
| 10 mM dNTP | 1 | μl |
| 10X Taq PCR buffer | 5 | μl |
| Taq polymerase | 1 | μl |
| dH₂O | 40 | μl |
| Total | 50 | μl |

First set in 95° C. to denature for 5 minutes, and then 25-30 cycles of 94° C. denature 30 seconds, 54° C. annealing 30 seconds, 72° C. elongation 35 seconds, and after 25-30 cycles, the final 72° C. for 10 minutes.

7. Analysis of PCR Products:

Load about 20 μl of PCR products to 1.5-2.0% agarose gel for electrophoresis check. The ssPCR result: the lane3, 6, 9, 11, 12, 13 are positive and lane 4, 5, 7, 8, 10 are negative which similar with ELISA analysis. Following is ELISA result:

| | 1 | 2 | 3 | 4 | 5 | 6 | 7 | 8 | 9 | 10 | 11 | 12 |
|---|---|---|---|---|---|---|---|---|---|---|---|---|
| A | 0.005 | 1.853 | 1.241 | 0.012 | 0.025 | 2.213 | 0.053 | 0.004 | 0.876 | 0.022 | 0.785 | 0.941 |
| B | 1.235 | 0.002 | 0.000 | 0.000 | 0.000 | 0.000 | 0.000 | 0.000 | 0.000 | 0.000 | 0.000 | 0.000 |

The A1 is negative control, A2 is positive control, A3-12 and 1B(13) are samples.

### Example 2

### Analysis of HBV YMDD Mutation

During hepatitis long term treatment with lamivudine, HBV developed drug-resistant mutation that HBV polymerase YMDD (tyrosine-methionine-aspartate-aspartate) motif changed either to YIDD or YVDD motif. The present art ssPCR is designed to detect the HBV drug-resistant point mutations which replaced with different size indicator DNA, YMDD motif with 300 bp indicator DNA1, YIDD with 200 bp indicator DNA2, and YVDD with 150 bp indicator DNA 3. Then reverse PCR of indicator DNA1, 2, and 3 will give 300 bp, 200 bp and 150 bp fragments in the agarose gel. The HBV polymerase partial sequence included YMDD (/YIDD/YVDD) motif was cloned to pUC₁₉ for producing pUC-YMDD (531-582aa), pUC-YIDD, and pUC-YVDD as positive control. The pUC₁₉ alone is as negative control.

### 1. Purification of the Test Sample:

The DNA samples of 6 chronic HBV infections with drug-resistant and 1 normal blood are first extracted once with equal of phenol/chloroform/isopentyl alcohol (25:24:1) reagent and once with chloroform alone to remove the proteins.
(1) Take test sample in the 100 μl volume in EP tube.
(2) Add 100 μl of phenol/chloroform/isopentyl alcohol mix, vortex.
(3) Spin 1500 rpm×5 minutes in micro-centrifuge.
(4) Transfer supernatant to a fresh tube, add 100 μl of chloroform and vortex.
(5) Spin 5 minutes at high speed in microcentrifuge.

The supernatant DNA further purification is accomplished by using Qiagen Inc DNA purification column kit according the operation menu.

### 2. Preparation of Indicator DNA:

(1) Select HBV polymerase - - - s Y [symbol] D D v - - - sequence 5′-c tgt t/c tg gct ttc agt tat [symbol] gat gat gtg gt w(a/t) ttg g-3′ as YMDD Preserved Hybridization Region (PHR); the mutant - - - s Y [symbol] D D v - - - sequence 5′-c tgt t/c tg gct ttc agt tat [symbol] gat gat gtg gtw(a/t) ttg g-3′ as YIDD PHR; the mutant - - - s Y [symbol] D D v - - - sequence 5′-c tgt t/c tg gct ttc agt tat [symbol] gat gat gtg gtw(a/t) ttg g-3′ as YVDD PHR.

(2) Select parasite Gluthione S-Transferase (GST13-341/aa5-114) is about 300 bp as YMDD indicator DNA1; the shorter same sequence which 5′end and 3′end both deleted about 50 bp (GST83-270 nt) is about 200 bp as YIDD indicator DNA2; more shorter same sequence which 5′end and 3′end both deleted more 25 bp (GST105-250 nt) is about 150 bp as YVDD indicator DNA3.

(3) Synthesis of Indicator 5′end Primers:

YMDD 5′end primer MF: the 18 base sense-chain sequence of right part YMDD PHR plus the 22 base sense-chain sequence of indicator DNA1 upstream 5′end.

5′-tg gat gat gtg gtW ttg gta ggt tat tgg aaa att
aag g-3′

YIDD 5′end primer IF: the 17 base sense-chain sequence of right part YIDD PHR plus the 19 base sense-chain sequence of indicator DNA2 upstream 5′end.

5′-t gat gat gtg gtW ttg gaa gag cat ttg tat gag
c-3′

YVDD 5′end primer VF: the 17 base sense-chain sequence of right part YVDD PHR plus the 19 base sense-chain sequence of indicator DNA3 upstream 5′end.

5′-g gat gat gtg gtW ttg gat gaa ggt gat aaa
tgg-3′

(4) Synthesis of Indicator 3′end Primers:

YMDD 3′end primer MR: the 20 base anti-sense sequence of left part YMDD PHR plus the 20 base anti-sense sequence of indicator DNA1 downstream 3′end.

5′-t ata act gaa agc caR aca gtc ttt act ata tgc
aat tc-3′

YIDD 3′end primer IR: the 21 base anti-sense sequence of left part YIDD PHR plus the 18 base anti-sense sequence of indicator DNA2 downstream 3′end.

5′-at ata act gaa agc caR aca gtc tgc acg ctc ttt
tgg-3′

YVDD 3′end primer VR: the 21 base anti-sense sequence of left part YVDD PHR plus the 17 base anti-sense sequence of indicator DNA3 downstream 3′end.

(5) Amplification and Purification of indicator DNA with cap and tail

| | | |
|---|---|---|
| pGEX-2T plasmid DNA | 1 | μl |
| (which part GST sequence as indicator) | | |
| Forward primer(5 μM) | 1 | μl |
| Reverse primer(5 μM) | 1 | μl |
| 10 mM dNTP | 1 | μl |
| 10X pfu buffer | 5 | μl |
| pfu | 1 | μl |
| dH₂O | 40 | μl |
| Total | 50 | μl |

First set in 95° C. to denature for 5 minutes, and then 25 cycles of 94° C. 30 seconds, 54° C. annealing 30 seconds, 72° C. elongation 35 seconds, and after 25 cycles, the final 72° C. for 10 minutes.

The PCR products are loaded to the 1.5%-2.0% agarose gel for gel purification by using Qiagen Inc kit. Final indicator DNA elute in 50 μl dH₂O.

3. Synthesis of Double-Strands Oligo (with Blunt End) Inhibitor:

Take extreme rare sequence-Homing Endonucleases I-Ceu I partial recognition site 5′-ggt cct aag gta gcg-3′ and complement anti-sense: 5′-cgc tac ctt agg acc-3′ as Oligo Inhibitor, combine them, 90° C. heat 5 minutes and cool at Room-Temperature, store at −20° C.

### 4. Synthesis of Reverse Primers

Take the same center region of indicator DNA1, 2, 3 about 40 bp sequence as the common reverse primer. The right half of sense-chain: 5′-at ggt gat gtt aaa tta aca c-3′ as common 5′end reverse primer RevF; the left half of anti-sense: 5′-atc aat ata ata agg aag att g-3′ as common 3′end reverse primer RevR.

### 5. Hybridization-Ligation Reaction of the Test-Indicator:

| | | |
|---|---|---|
| Purified test sample | 1 | μl |
| Purified indicator with cap & tail | 1 | μl |
| 10X Ligase buffer | 2 | μl |
| Oligo inhibitor (0.1 mM) | 1 | μl |
| Thermus Ligase(such as Taq ligase) | 0.5-1 | μl |
| dH₂O | 14 | μl |
| Total | 20 | μl |

Set in 95° C. to denature for 5 minutes, and then in 50° C. (40-70° C.) hybridization-ligation for 10 minutes.

### 6. Reverse PCR of the Circular Indicator:

Use the solution of hybridization-ligation reaction of the test-indicator as the reverse PCR template and the center sequences of indicator (Reverse primer F, Reverse primer R) as the reverse PCR primers.

| | | |
|---|---|---|
| Solution of hybridization-ligation | 2 | μl |
| of the test-indicator | | |
| 5′end Reverse pimer RevF(5 uM) | 1 | μl |
| 3′end Reverse pimer RevR(5 uM) | 1 | μl |
| 10 mM dNTP | 1 | μl |
| 10X Taq PCR buffer | 5 | μl |
| Taq polymerase | 1 | μl |
| dH₂O | 40 | μl |
| Total | 50 | μl |

First set in 95° C. to denature for 5 minutes, and then 25-30 cycles of 94° C. denature 30 seconds, 54° C. annealing 30 seconds, 72° C. elongation 35 seconds, and after 25-30 cycles, the final 72° C. for 10 minutes.

### 7. Analysis of PCR Products:

Load about 20 μl of PCR products to 1.5-2.0% agarose gel for electrophoresis check. The result of ssPCR: The lane1 is normal blood, lane 2, 3, 4 are the YMDD HBV infections, lane 5 is YMDD and YIDD mix infection, lane 6 is YIDD alone infection, lane 7 is YVDD infection, lane 8 is pUC₁₉ alone negative control and lane 9 is positive control. The YMDD positive samples show 300 bp fragment, YIDD's show 200 bp fragment and YVDD's show 150 bp fragment. The YIDD and YVDD sample was cloned and sequenced by commercial service. The sequence result confirmed the M to I/V mutations.

1. A method of substituting the test PCR with indicator DNA amplification involving the test DNA-indicator substitution or translation by using test-related indicator preparation comprising adding part test sequence to two ends of indicator following indicator ends associate with test DNA wherein the part test DNA sequence is chosen as Preserved Hybridization Region (PHR), in which the right half PHR is linked with upstream (5′) end of indicator DNA as cap, and the left half PHR is attached to downstream (3′) end of indicator DNA as tail wherein these indicator DNA with cap and tail that have test Preserved Hybridization Region can associate with test template through their complementary cap and tail sequences the method further comprising a test-indicator hybridization ligation reaction wherein by the test template association help, the indicator DNA cap end will close to tail termini and form hybrids of the cap and tail sequence of indicator with test strands and wherein a nick between cap and tail ends of hybrids is joined by ligase such that the indicator that associated with test becomes a circular DNA and the method further comprising reverse amplification of the circular indicator DNA comprising reverse PCR of the circular indicator DNA by using center sequence of the indicator as reverse primers will replace the direct test PCR.

2. The method according to claim 1, wherein the test DNA is substituted with a series of different, independent indicator DNAs comprising, if a first indicator DNA PCR is contaminated, changing to a different indicator DNA system.

3. The method according to claim 1, wherein the test DNA is double stranded DNA, or DNA-RNA hybrids, or RNA, or mRNA, wherein the Preserved Hybridization Region (PHR) of test is a 20-200 bp length conserved sequence.

4. The method according to claim 3, wherein the length of test PHR is a 30-50 base-pair sequence.

5. The method according to claim 1, wherein the length of indicator DNA is a 80-1000 bp irrelevance sequence with test, and plus the right PHR to upstream end as cap and the left PHR to downstream end as tail.

6. The method according to claim 5, wherein the length of indicator DNA is a 80-150 bp sequence.

7. The method according to claim 1, wherein the Ligase is a DNA ligase selected from among T4 ligase, and Taq ligase.

8. The method according to claim 1, wherein the System Substitute PCR (ssPCR) further comprises Real-Time Fluorescent PCR which test sequence specific fluorescent probes substituted with indicator sequence specific fluorescent probes.

9. The method according to claim 8, wherein the Real-Time PCR utilizes DNA dye SYBR Green dye, and fluorescent probes selected from among Molecular Beacon, Taqmen, and Light Cycler.

10. The method according to claim 1, wherein the method comprises detecting single base substitution or genetic mutation of genes.

11. The method according to claim 2, wherein the method comprises detecting single base substitution or genetic mutation of genes.

12. The method according to claim 3, wherein the method comprises detecting single base substitution or genetic mutation of genes.

13. The method according to claim 4, wherein the method comprises detecting single base substitution or genetic mutation of genes.

14. The method according to claim 5, wherein the method comprises detecting single base substitution or genetic mutation of genes.

15. The method according to claim 6, wherein the method comprises detecting single base substitution or genetic mutation of genes.

16. The method according to claim 7, wherein the method comprises detecting single base substitution or genetic mutation of genes.

17. The method according to claim 8, wherein the method comprises detecting single base substitution or genetic mutation of genes.

18. The method according to claim 9, wherein the method comprises detecting single base substitution or genetic mutation of genes.
